(19) United States
(12) Patent Application Publication
(10) Pub. No.: US 2017/0273948 A1
(43) Pub. Date: Sep. 28, 2017

(54) USE OF SIGMA RECEPTOR LIGANDS IN OSTEOARTHRITIS

(71) Applicant: LABORATORIOS DEL DR ESTEVE S.A., Barcelona (ES)

(72) Inventors: José Miguel VELA HERNÁNDEZ, Barcelona (ES); Manuel MERLOS-ROCA, BARCELONA (ES); Daniel ZAMANILLO-CASTANEDO, Barcelona (ES)

(21) Appl. No.: 15/532,168

(22) PCT Filed: Dec. 15, 2015
(86) PCT No.: PCT/EP2015/002524

§ 371 Date: Jun. 1, 2017

(30) Foreign Application Priority Data
Dec. 15, 2014 (EP) 14382519.8
Jan. 28, 2015 (EP) 15000261.6

(51) Int. Cl. A61K 31/415; A61K 31/5377; A61K 31/496

(52) U.S. Cl. CPC A61K 31/415; A61K 31/496; A61K 31/5377; A61K 45/06

(57) ABSTRACT

The invention relates to compounds of formula (I) or formula (II)

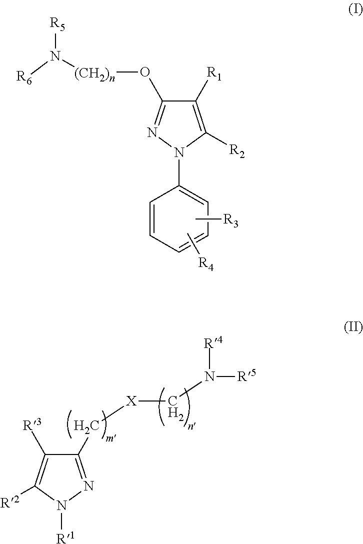

having pharmacological activity towards the sigma receptor, for use in the treatment or prevention of osteoarthritis and pain due to osteoarthritis.

### FIELD OF THE INVENTION

The present invention relates to the use of sigma receptor ligands, and more particularly to some pyrazole derivatives, to pharmaceutical compositions comprising them, and to their use for the treatment or prevention of osteoarthritis and pain due to osteoarthritis.

### BACKGROUND OF THE INVENTION

Osteoarthritis (OA) is the most common condition to affect synovial joints, the single most important cause of locomotor disability, and a major challenge to health care, affecting growing numbers of people in ageing populations (Jones & Doherty, Br Med J, 1995, 310, 457). It is estimated that more than 20 million Americans and 35 to 40 million Europeans suffer from OA (Mobasheri, Curr Rheumatol Rep, 2013, 15, 364). OA affects at least 50% of people>65 years of age, and occurs in younger individuals following joint injury. The societal burden (both in terms of personal suffering and use of health resources) is expected to increase with the increasing prevalence of obesity and the ageing of the population (Hunter & Felson, Br Med J, 2006, 332, 639).

OA is characterised by focal cartilage loss and an accompanying reparative bone response. The commonest joints affected are large weight-bearing joints, such as the hip and knee, and smaller peripheral joints, including the hands (Sofat et al., Rheumatology, 2011, 50, 2157). OA is fundamentally different in terms of pathogenesis, prognosis, and medical management from rheumatoid arthritis, another common arthritic condition which is characterised by inflammation and autoimmune response (Ravi et al., Arthritis Rheumatism, 2012, 64, 3839). The diagnosis of OA can usually be made clinically and then confirmed by radiography. The main features that suggest the diagnosis include pain, stiffness, reduced movement, swelling, crepitus and increased age in the absence of systemic features (Hunter & Felson, Br Med J, 2006, 332, 639).

Pain is the main reason for presentation of OA patients to clinical services. Patients largely present with pain and disability after significant loss of cartilage has occurred, but it is estimated that up to 40% of individuals with radiological damage have no pain (Sofat et al., 2011). There are currently no treatments that are known to modify disease progression. At present, licensed treatments for OA are focused on the relief of pain symptoms and other physical treatments aiming to improve function such as physiotherapy and rehabilitation, as well as surgical joint replacement in suitable individuals (Sofat & Kuttapitiya, Int J Rheumatol, 2014, 9, 197). Many people with OA continue to suffer from pain symptoms despite currently available treatments. There is, therefore, an unmet need to develop more efficient analgesic agents (Sofat et al., 2011).

Since the most common joints affected by OA are large weight-bearing joints such as hip and knee, intra-knee injection of the chondrocyte glycolytic inhibitor mono-iodoacetate (MIA) OA models, as well as surgically induced-, and spontaneous knee OA models have been used to investigate mechanisms of OA-induced pain (Zhang et al., Osteoarthritis Cartilage, 2013, 21, 1308).

The challenges in the clinical treatment of OA are daunting as many therapeutic regimes solely rely on the treatment of symptoms, mostly by change in lifestyle (e.g. weight loss), physical exercise and administering analgesics against pain. However, a recent review found that the three major groups of analgesics, NSAIDs, opioids and COX-2 inhibitors, used in the treatment of rheumatoid arthritis (RA) and Osteoarthritis (OA) display significant side effects (Solomon et al., Arch Intern Med, 2010, 170, 1968).

Importantly, as OA is caused by degeneration of cartilage (and subchondral bone) and OA pain results from activation of subchondral nociceptive fibers and subsequent amplification (sensitization) of nociceptive pathways, it would be crucial to identify therapeutic agents that can stop or slow down the progression of the disease and/or the sensitization of pain pathways, that is, e.g. to inhibit and/or prevent cartilage loss/degeneration (or inhibit/prevent additional/further degeneration) and/or associated pain, in OA patients. However, at the moment such “Disease Modifying Drugs” for OA (DMOAD) have not been identified.

We have found two families of structurally distinct pyrazol derivatives which are particularly selective inhibitors of the sigma receptor.

The first family presents a pyrazol group which is characterized by the substitution at position 3 by an alkoxy group directly bounded to a nitrogen. These compounds have been described in WO 2006/021462, and have also been characterized in WO 2009/103487, WO 2009/130310, WO 2011/018487, WO 2011/095585, WO 2011/144721, WO 2011/095584, WO 2012/016980, WO 2012/072782, WO 2012/156497, WO 2006/010587, WO 2007/025613.

The second family presents a pyrazol group which is characterized by the substitution at position 3 by an alkyl chain containing an amine at its end and optionally an intermediate oxa moiety. These compounds have been described in WO 2011/147910.

The sigma (σ) receptor is a cell surface and endoplasmic reticulum receptor expressed in the central nervous system (CNS) among other tissues. From studies of the biology and function of sigma receptors, evidence has been presented that sigma receptor ligands may be useful in the treatment of psychosis and movement disorders such as dystonia and tardive dyskinesia, and motor disturbances associated with Huntington's chorea or Tourette's syndrome and in Parkinson's disease (Walker, J. M. et al, Pharmacological Reviews, 1990, 42, 355). It has been reported that the known sigma receptor ligand rimcazole clinically shows effects in the treatment of psychosis (Hanner, M. et al. Proc. Natl. Acad. Sci., 1996, 93:8072-8077; Snyder, S. H., Largent, B. L. J. Neuropsychiatry 1989, 1, 7). The sigma binding sites have preferential affinity for the dextrorotatory isomers of certain opiate benzomorphans, such as (+)SKF 10047, (+)cyclazocine, and (+)pentazocine and also for some narcoleptics such as haloperidol.

The sigma receptor has at least two subtypes, which may be discriminated by stereoselective isomers of these pharmacoactive drugs. SKF 10047 has nanomolar affinity for the sigma 1 (σ-1) site, and has micromolar affinity for the sigma (σ-2) site. Haloperidol has similar affinities for both subtypes. Endogenous sigma ligands are not known, although progesterone has been suggested to be one of them. Possible sigma-site-mediated drug effects include modulation of glutamate receptor function, neurotransmitter response, neuroprotection, behavior, and cognition (Quirion, R. et al. Trends Pharmacol. Sci., 1992, 13:85-86). The existence of sigma receptors in the CNS, immune and endocrine systems have suggested a likelihood that it may serve as link between the three systems.

It was surprisingly found that these sigma-receptor ligands of the invention have a dual function in that they are effective as analgesics against pain due to OA and they also exert a disease-modifying effect as they progressively ameliorate OA pain (progressive restoration back to normal of baseline pain thresholds) following repeated administrations.

Therefore, the present invention provides sigma-receptor ligands that are both, effective in the treatment or prevention of osteoarthritis (DMOAD-activity), and effective in the treatment or prevention of pain due to osteoarthritis.

### SUMMARY OF THE INVENTION

In one aspect the invention is directed to a compound binding to the sigma-receptor for use in the treatment or prevention of osteoarthritis, and/or for the treatment or prevention of pain due to osteoarthritis.

In a preferred embodiment of the use as defined above the pain is selected from acute and/or chronic pain due to osteoarthritis, especially neuropathic pain, neuralgia, allodynia, causalgia, hyperalgesia, hyperesthesia, hyperpathia, neuritis or neuropathy secondary to surgical procedure.

In a preferred embodiment of the use as defined above the compound is selected from a sigma receptor antagonist, a neutral antagonist, an inverse agonist or a partial antagonist.

In a preferred embodiment of the use as defined above the compound binds to the sigma-1 receptor subtype.

In a preferred embodiment of the use as defined above the compound is a compound according to formula I:

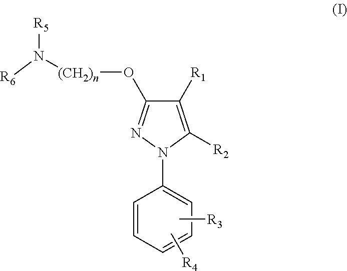

wherein
R₁ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted non-aromatic heterocyclyl, substituted or unsubstituted aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen;
R₂ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen;
R₃ and R₄ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen, or together they form an optionally substituted fused ring system;
R₅ and R₆ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)ₜ—R₈, —NR₈R₉, —NR₈C(O)R₉, —NO₂, —N═CR₈R₉, and halogen, or together form, with the nitrogen atom to which they are attached, a substituted or unsubstituted, aromatic or non-aromatic heterocyclyl group;
   n is selected from 1, 2, 3, 4, 5, 6, 7 or 8;
   t is 1, 2 or 3;
R₈ and R₉ are each independently selected from hydrogen, substituted or unsubstituted alkyl, substituted or unsubstituted cycloalkyl, substituted or unsubstituted alkenyl, substituted or unsubstituted aryl, substituted or unsubstituted, aromatic or non-aromatic heterocyclyl, substituted or unsubstituted alkoxy, substituted or unsubstituted aryloxy, and halogen;
   or a pharmaceutically acceptable salt, isomer, prodrug or solvate thereof, or preferably a pharmaceutically acceptable salt or isomer thereof.

In another preferred embodiment of the use as defined above the compound is a compound according to formula II:

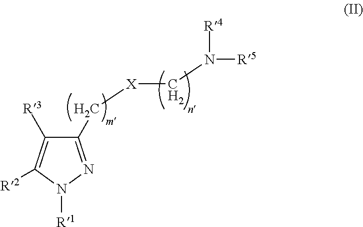

wherein
R′¹ represents substituted or unsubstituted aromatic or non-aromatic heterocyclyl; substituted or unsubstituted aryl; or substituted or unsubstituted cycloalkyl;
R′² and R′³, identical or different, represent a hydrogen atom; F; Cl; Br; I; CF₃; OH; SH; NH₂; CN; substituted or unsubstituted alkyl; substituted or unsubstituted alkenyl; substituted or unsubstituted alkoxy; substituted or unsubstituted cycloalkyl; substituted or unsubstituted aryl; substituted or unsubstituted, aromatic or non-aromatic heterocyclyl; substituted or unsubstituted cycloalkylalkyl; substituted or unsubstituted arylalkyl; substituted or unsubstituted, aromatic or non-aromatic heterocyclylalkyl; a (C═O)—R′⁷ group; a (C═O)—O—R′⁸ group; a S(O)_{t′}—R′⁹ group; or a (C═O)—NR′¹⁰R′¹¹ group;
R′⁴ and R′⁵, identical or different, represent a hydrogen atom; substituted or unsubstituted alkyl; substituted or unsubstituted alkenyl; substituted or unsubstituted alkoxy; substituted or unsubstituted cycloalkyl; substituted or unsubstituted aryl; substituted or unsubstituted, aromatic or non-aromatic heterocyclyl; substituted or unsubstituted cycloalkylalkyl; substituted or unsubstituted arylalkyl; substituted or unsubstituted, aromatic or non-aromatic heterocyclylalkyl; a (C═O)—R′⁷ group; a (C═O)—O—R′⁸ group; a S(O)_{t′}—R′⁹ group; or a (C═O)—NR′¹⁰R′¹¹ group;
or
together form, with the nitrogen atom to which they are attached, a substituted or unsubstituted, aromatic or non-aromatic heterocyclyl group;
X represents an oxygen atom or a CH—R′¹² group wherein R′¹² is selected from H, CH₃, SH, OH, NH₂, CF₃, Cl, F, Br, I, and CN;
m′ is selected from 1, 2, 3 and 4;
n′ is selected from 1, 2, 3 and 4;
t′ is selected from 1, 2 and 3;
R′⁷, R′⁸, R′⁹, R′¹⁰ and R′¹¹, identical or different, represent a hydrogen atom; substituted or unsubstituted C₁₋₆ alkyl; substituted or unsubstituted C₁₋₆ alkenyl; substituted or unsubstituted C₁₋₆ alkoxy; substituted or unsubstituted cycloalkyl; substituted or unsubstituted aryl; substituted or unsubstituted, aromatic or non-aromatic heterocyclyl; substituted or unsubstituted cycloalkylalkyl; substituted or unsubstituted arylalkyl; substituted or unsubstituted, aromatic or non-aromatic heterocyclylalkyl;
   or a pharmaceutically acceptable salt, isomer, prodrug or solvate thereof or preferably a pharmaceutically acceptable salt or isomer thereof.

In another aspect the invention is directed to a pharmaceutical composition comprising a compound as defined above, wherein the composition further comprises a pharmaceutically acceptable carrier, adjuvant and/or vehicle.

The above mentioned preferences and embodiments can be combined to give further preferred compounds or uses.

### BRIEF DESCRIPTION OF THE FIGURES

FIG. 1 illustrates to the experimental protocol of Example A.

FIG. 2 shows the effect of the compound of example 1 and Tramadol on a rat model of osteoarthritis, wherein mechanical allodynia was evaluated to measure pain due to osteoarthritis and evaluate pain relieving effects of compounds.

FIG. 3 illustrates to the experimental protocol of Example B.

FIG. 4 shows the effect of the compound of example 3 and Oxycodone on a rat model of osteoarthritis, wherein mechanical allodynia was evaluated to measure pain due to osteoarthritis and evaluate pain relieving effects of compounds.

### DETAILED DESCRIPTION OF THE INVENTION

In one aspect the invention is directed to a compound binding to the sigma-receptor for use in the treatment or prevention of osteoarthritis, and/or for the treatment or prevention of pain due to osteoarthritis.

The term “Osteoarthritis” refers to any kind of cartilage loss and subchondral bone degeneration/damage in any kind of (bone-) joints in the body of a mammal. Preferably, it refers to arthritic changes of the larger and the smaller joints of the body, including the hands, wrists, feet, back, hip, and knee.

The term “treatment and/or prevention of osteoarthritis” refers to any kind of inhibitory effect of the compounds of the invention regarding the loss/degeneration of cartilage and/or subchondral bone in Osteoarthritis as defined above.

The term “compound binding to the sigma receptor” refers to any compound that binds with high affinity to the sigma-receptor, preferably to the sigma-1 receptor subtype.

The expression “binding with high affinity to the sigma receptor” refers to compounds of the invention that can replace a ligand in competitive binding assays, preferably in competitive radioligand-binding assays as exemplary described in WO2006/021462, e.g. in binding assays for the al-receptor performed as described (DeHaven-Hudkins et al., Eur J Pharmacol, 1992, 227, 371) or binding assays for σ2-receptor as described (Radesca et al., J Med Chem, 1991, 34, 3058). Preferably, binding of the compounds of the invention, with respect to binding to the sigma-1 receptor subtype, is measured by competing with the binding of ³[H]-(+)-pentazocine, e.g. in radioligand-assays as described in the art (e.g. in DeHaven-Hudkins et al., 1992). Preferably, compounds of the invention when assayed at a concentration of 10⁻⁷M yield at least 25%, more preferably at least 45%, even more preferably at least 65%, yet even more preferably at least 75%, most preferably at least 85% binding to the sigma-1 receptor in ³[H]-(+)-pentazocine radioligand-assays as defined above.

When comparing to current pharmaceutical compounds used in the treatment of OA (i.e. Tramadol), and-entirely unexpected, the efficacy of Sigma ligands according to the invention in a repeated daily treatment of OA is significantly higher than that of conventional compounds. On the one side, considering the data presented in FIG. 2, it is evident that the compounds of the invention show an immediate analgesic effect (comparing “PRE” and “POST” values of the same day). Surprisingly, the Sigma ligands as defined herein—but not the compounds currently used in treatment of OA—progressively ameliorate the basal pain found before daily treatments, which is consistent with a modification of mechanisms underlying pain (comparing “PRE” values over time during treatment).

Further, the observed results demonstrate that Sigma ligands according to the invention do not induce analgesic tolerance (loss of analgesic efficacy following repeated treatment). On the contrary, as discussed above, they show a surprising increase of activity with time following its repeated administration, which is associated with a progressive restoration back to normal of baseline nociceptive thresholds and strongly points to a disease-modifying effect. The disease-modifying effect exerted by the repeated treatment with the Sigma ligands above defined was also evidenced after treatment discontinuation. In fact it was indeed observed that treatment with the Sigma ligands according to the invention is required not only to modify the disease but also to maintain the modification.

Importantly, as shown herein, the compounds of the present invention display a long-term pain-improving effect and mobility-improving effect on test animals, most likely through preventing, inhibiting and/or interfering with cartilage degeneration (and subchondral bone degeneration). Thus the compounds of the invention can slow down or even stop disease progression in Osteoarhtritis and therefore act as “Disease modifying Osteoarthritis Drugs” (DMOADs).

Therefore, another aspect of the invention is the use of the Sigma ligands as defined above as disease modifying drugs for OA and thus being useful for the treatment or prevention of osteoarthritis and/or pain due to osteoarthritis.

Thus, in a preferred embodiment the compound binding to the sigma-receptor is used in the treatment or prevention of osteoarthritis.

In a preferred embodiment the compound binding to the sigma-receptor is used in the treatment of osteoarthritis.

Further, the DMOAD-activity of the compounds of the invention clearly makes them an interesting tool in the prevention of osteoarthritis.

In a preferred embodiment the compound binding to the sigma-receptor is used in the prevention of osteoarthritis.

As is shown below, the compounds of the invention demonstrate high analgesic efficacy regarding pain due to osteoarthritis.

In a preferred embodiment the compound binding to the sigma-receptor is used in the treatment or prevention of pain due to osteoarthritis.

In a preferred embodiment the compound binding to the sigma-receptor is used in the treatment of pain due to osteoarthritis.

In a preferred embodiment the compound binding to the sigma-receptor is used in the prevention of pain due to osteoarthritis.

In a preferred embodiment of the use as defined above the pain is selected from acute and/or chronic pain due to osteoarthritis, especially neuropathic pain, neuralgia, allodynia, causalgia, hyperalgesia, hyperesthesia, hyperpathia, neuritis or neuropathy secondary to surgical procedure.

In a preferred embodiment of the use as defined above the compound is selected from a sigma receptor antagonist, a neutral antagonist, an inverse agonist or a partial antagonist.

In a preferred embodiment of the use as defined above the compound binds to the sigma-1 receptor subtype.

In a preferred embodiment of the use as defined above the compound is a compound according to formula I:

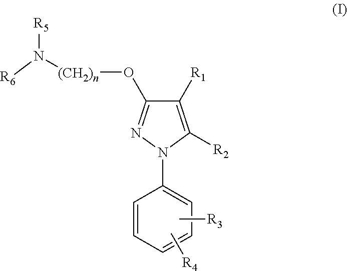

wherein
R₁ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted non-aromatic heterocyclyl, substituted or unsubstituted aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen;
R₂ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen;
R₃ and R₄ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen, or together they form an optionally substituted fused ring system;
R₅ and R₆ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen, or together form, with the nitrogen atom to which they are attached, a substituted or unsubstituted, aromatic or non-aromatic heterocyclyl group;
   n is selected from 1, 2, 3, 4, 5, 6, 7 or 8;
   t is 1, 2 or 3;
R₈ and R₉ are each independently selected from hydrogen, substituted or unsubstituted alkyl, substituted or unsubstituted cycloalkyl, substituted or unsubstituted alkenyl, substituted or unsubstituted aryl, substituted or unsubstituted, aromatic or non-aromatic heterocyclyl, substituted or unsubstituted alkoxy, substituted or unsubstituted aryloxy, and halogen;
   or a pharmaceutically acceptable salt, isomer, prodrug or solvate thereof, or preferably a pharmaceutically acceptable salt or isomer thereof.

In a preferred embodiment of the use as defined above the compound is characterized in that R₁ selected from H, —COR₈, or substituted or unsubstituted alkyl, preferably it is selected from H, methyl or acetyl.

In a preferred embodiment of the use as defined above the compound is characterized in that R₁ is hydrogen.

In a preferred embodiment of the use as defined above the compound is characterized in that R₂ is H or alkyl, preferably methyl or H.

In a preferred embodiment of the use as defined above the compound is characterized in that R₃ and R₄ are situated in the meta and para positions of the phenyl group.

In a preferred embodiment of the use as defined above the compound is characterized in that R₃ and R₄ are independently selected from halogen, or substituted or unsubstituted alkyl, more preferably selected from halogen or haloalkyl.

In an especially preferred embodiment of the use as defined above the compound is characterized in that both R₃ and R₄ together with the phenyl group form an optionally substituted fused ring system. More preferably, said fused ring system is selected from a substituted or unsubstituted fused aryl group and a substituted or unsubstituted aromatic or partially aromatic fused heterocyclyl group. Said fused ring system preferably contains two rings and/or from 9 to about 18 ring atoms, more preferably 9 or 10 ring atoms. Even more preferably, the fused ring system is naphthyl, especially a 2-naphthyl ring system, substituted or unsubstituted.

In a preferred embodiment of the use as defined above the compound is characterized in that n is selected from 2, 3, 4, more preferably n is 2.

In a preferred embodiment of the use as defined above the compound is characterized in that R₅ and R₆, together, form a morpholine-4-yl group.

In a preferred variant of the invention the sigma ligand of general formula (I) is selected from:
[1] 4-{2-(1-(3,4-Dichlorophenyl)-5-methyl-1H pyrazol-3-yloxy)ethyl}morpholine,
[2] 2-[1-(3,4-Dichlorophenyl)-5-methyl-1H-pyrazol-3-yloxy]-N,N-diethylethanamine hydrochloride
[3] 1-(3,4-Dichlorophenyl)-5-methyl-3-[2-(pyrrolidin-1-yl)ethoxy]-1H-pyrazole hydrochloride
[4] 1-(3,4-Dichlorophenyl)-5-methyl-3-[3-(pyrrolidin-1-yl)propoxy]-1H-pyrazole hydrochloride
[5] 1-{2-[1-(3,4-Dichlorophenyl)-5-methyl-1H-pyrazol-3-yloxy]ethyl}piperidine
[6] 1-{2-[1-(3,4-dichlorophenyl)-5-methyl-1H-pyrazol-3-yloxy]ethyl}-1H-imidazole
[7] 3-{1-[2-(1-(3,4-Dichlorophenyl)-5-methyl-1H-pyrazol-3-yloxy)ethyl]piperidin-4-yl}-3H-imidazo[4,5-b]pyridine
[8] 1-{2-[1-(3,4-Dichlorophenyl)-5-methyl-1H-pyrazol-3-yloxy]ethyl}-4-methylpiperazine
[9] Ethyl 4-{2-[1-(3,4-dichlorophenyl)-5-methyl-1H-pyrazol-3-yloxy]ethyl}piperazine carboxylate
[10] 1-(4-(2-(1-(3,4-dichlorophenyl)-5-methyl-1H-pyrazol-3-yloxy)ethyl)piperazin-1-yl)ethanone
[11] 4-{2-[1-(4-Methoxyphenyl)-5-methyl-1H-pyrazol-3-yloxy]ethyl}morpholine hydrochloride
[12] 1-(4-Methoxyphenyl)-5-methyl-3-[2-(pyrrolidin-1-yl)ethoxy]-1H-pyrazole
[13] 1-(4-Methoxyphenyl)-5-methyl-3-[3-(pyrrolidin-1-yl)propoxy]-1H-pyrazole
[14] 1-[2-(1-(4-Methoxyphenyl)-5-methyl-1H-pyrazol-3-yloxy)ethyl]piperidine
[15] 1-{2-[1-(4-Methoxyphenyl)-5-methyl-1H-pyrazol-3-yloxy]ethyl}-1H-imidazole
[16] 4-{2-[1-(3,4-Dichlorophenyl)-5-phenyl-1H-pyrazol-3-yloxy]ethyl}morpholine hydrochloride
[17] 1-(3,4-Dichlorophenyl)-5-phenyl-3-[2-(pyrrolidin-1-yl)ethoxy]-1H-pyrazole hydrochloride
[18] 1-(3,4-Dichlorophenyl)-5-phenyl-3-[3-(pyrrolidin-1-yl)propoxy]-1H-pyrazole
[19] 1-{2-[1-(3,4-Dichlorophenyl)-5-phenyl-1H-pyrazol-3-yloxy]ethyl}piperidine
[20] 1-{2-[1-(3,4-Dichlorophenyl)-5-phenyl-1H-pyrazol-3-yloxy]ethyl}-1H-imidazole hydrochloride
[21] 2-{2-[1-(3,4-dichlorophenyl)-5-phenyl-1H-pyrazol-3-yloxy]ethyl}-1,2,3,4-tetrahydroisoquinoline hydrochloride
[22] 4-{4-[1-(3,4-Dichlorophenyl)-5-methyl-1H-pyrazol-3-yloxy]butyl}morpholine hydrochloride
[23] 1-(3,4-Dichlorophenyl)-5-methyl-3-[4-(pyrrolidin-1-yl)butoxy]-1H-pyrazole
[24] 1-{4-[1-(3,4-Dichlorophenyl)-5-methyl-1H-pyrazol-3-yloxy]butyl}piperidine hydrochloride
[25] 1-{4-[1-(3,4-Dichlorophenyl)-5-methyl-1H-pyrazol-3-yloxy]butyl}-4-methylpiperazine dihydrochloride
[26] 1-{4-[1-(3,4-Dichlorophenyl)-5-methyl-1H-pyrazol-3-yloxy]butyl}-1H-imidazole
[27] 4-[1-(3,4-Dichlorophenyl)-5-methyl-1H-pyrazol-3-yloxy]-N,N-diethylbutan-1-amine
[28] 1-{4-[1-(3,4-dichlorophenyl)-5-methyl-1H-pyrazol-3-yloxy]butyl}-4-phenylpiperidine hydrochloride
[29] 1-{4-[1-(3,4-dichlorophenyl)-5-methyl-1H-pyrazol-3-yloxy]butyl}-6,7-dihydro-1H-indol-4(5H)-one
[30] 2-{4-[1-(3,4-dichlorophenyl)-5-methyl-1H-pyrazol-3-yloxy]butyl}-1,2,3,4-tetrahydroisoqui-noline
[31] 4-{2-[1-(3,4-dichlorophenyl)-5-isopropyl-1H-pyrazol-3-yloxy]ethyl}morpholine hydrochloride
[32] 2-[1-(3,4-Dichlorophenyl)-5-isopropyl-1H-pyrazol-3-yloxy]-N,N-diethylethanamine
[33] 1-(3,4-Dichlorophenyl)-5-isopropyl-3-[2-(pyrrolidin-1-yl)ethoxy]-1H-pyrazole hydrochloride
[34] 1-(3,4-Dichlorophenyl)-5-isopropyl-3-[3-(pyrrolidin-1-yl)propoxy]-1H-pyrazole hydrochloride
[35] 1-{2-[1-(3,4-Dichlorophenyl)-5-isopropyl-1H-pyrazol-3-yloxy]ethyl}piperidine
[36] 2-{2-[1-(3,4-dichlorophenyl)-5-isopropyl-1H-pyrazol-3-yloxy]ethyl}-1,2,3,4-tetrahydroisoqui-noline hydrochloride
[37] 4-{2-[1-(3,4-dichlorophenyl)-1H-pyrazol-3-yloxy]ethyl}morpholine
[38] 2-[1-(3,4-dichlorophenyl)-1H-pyrazol-3-yloxy]N,N-diethylethanamine
[39] 1-(3,4-dichlorophenyl)-3-[2-(pyrrolidin-1-yl)ethoxy]-1H-pyrazole
[40] 1-{2-[1-(3,4-dichlorophenyl)-1H-pyrazol-3-yloxy]ethyl}piperidine
[41] 1-(3,4-dichlorophenyl)-3-[3-(pyrrolidin-1-yl)propoxy]-1H-pyrazole
[42] 1-{2-[1-(3,4-Dichlorophenyl)-5-methyl-1H-pyrazol-3-yloxy]ethyl}piperazine dihydrochloride
[43] 1-{2-[1-(3,4-Dichlorophenyl)-5-methyl-1H-pyrazol-3-yloxy]ethyl}pyrrolidin-3-amine
[44] 4-{2-[1-(3,4-Dichlorophenyl)-4,5-dimethyl-1H-pyrazol-3-yloxy]ethyl}morpholine
[45] 2-[1-(3,4-Dichlorophenyl)-4,5-dimethyl-1H-pyrazol-3-yloxy]-N,N-diethylethanamine hydrochloride
[46] 1-(3,4-Dichlorophenyl)-4,5-dimethyl-3-[2-(pyrrolidin-1-yl)ethoxy]-1H-pyrazole hydrochloride
[47] 1-(3,4-Dichlorophenyl)-4,5-dimethyl-3-[3-(pyrrolidin-1-yl)propoxy]-1H-pyrazole hydrochloride
[48] 1-{2-[1-(3,4-Dichlorophenyl)-4,5-dimethyl-1H-pyrazol-3-yloxy]ethyl}piperidine
[49] 4-{4-[1-(3,4-dichlorophenyl)-1H-pyrazol-3-yloxy]butyl}morpholine hydrochloride
[50] (2S,6R)-4-{4-[1-(3,4-dichlorophenyl)-1H-pyrazol-3-yloxy]butyl}-2,6-dimethylmorpholine hydrochloride
[51] 1-{4-[1-(3,4-Dichlorophenyl)-1H-pyrazol-3-yloxy]butyl}piperidine hydrochloride
[52] 1-(3,4-Dichlorophenyl)-3-[4-(pyrrolidin-1-yl)butoxy]-1H-pyrazole hydrochloride
[53] 4-[1-(3,4-dichlorophenyl)-1H-pyrazol-3-yloxy]-N,N-diethylbutan-1-amine oxalate
[54] N-benzyl-4-[1-(3,4-dichlorophenyl)-1H-pyrazol-3-yloxy]-N-methylbutan-1-amine oxalate
[55] 4-[1-(3,4-dichlorophenyl)-1H-pyrazol-3-yloxy]-N-(2-methoxyethyl)-N-methylbutan-1-amine oxalate
[56] 4-{4-[1-(3,4-dichlorophenyl)-1H-pyrazol-3-yloxy]butyl}thiomorpholine oxalate
[57] 1-[1-(3,4-Dichlorophenyl)-5-methyl-3-(2-morpholinoethoxy)-1H-pyrazol-4-yl]ethanone oxalate
[58] 1-{1-(3,4-dichlorophenyl)-5-methyl-3-[2-(pyrrolidin-1-yl)ethoxy]-1H-pyrazol-4-yl}ethanone oxalate
[59] 1-{1-(3,4-dichlorophenyl)-5-methyl-3-[2-(piperidin-1-yl)ethoxy]-1H-pyrazol-4-yl}ethanone oxalate
[60] 1-{1-(3,4-dichlorophenyl)-3-[2-(diethylamino)ethoxy]-5-methyl-1H-pyrazol-4-yl}ethanone oxalate
[61] 4-{2-[5-Methyl-1-(naphthalen-2-yl)-1H-pyrazol-3-yloxy]ethyl}morpholine
[62] N,N-Diethyl-2-[5-methyl-1-(naphthalen-2-yl)-1H-pyrazol-3-yloxy]ethanamine
[63] 1-{2-[5-Methyl-1-(naphthalen-2-yl)-1H-pyrazol-3-yloxy]ethyl}piperidine hydrochloride
[64] 5-Methyl-1-(naphthalen-2-yl)-3-[2-(pyrrolidin-1-yl)ethoxy]-1H-pyrazole hydrochloride
   their salts, different alternative pharmaceutically acceptable salts, solvates or prodrugs, preferably their salts, or different alternative pharmaceutically acceptable salts.

In a more preferred embodiment of the use as defined above the compound is 4-{2-[5-Methyl-1-(naphthalen-2-yl)-1H-pyrazol-3-yloxy]ethyl}morpholine or its pharmaceutically acceptable salts, solvates or a prodrug thereof. Preferably, the compound is 4-{2-[5-Methyl-1-(naphthalen-2-yl)-1H-pyrazol-3-yloxy]ethyl}morpholine or its pharmaceutically acceptable salts.

In a still more preferred embodiment of the use as defined above the compound is 4-{2-[5-Methyl-1-(naphthalen-2-yl)-1H-pyrazol-3-yloxy]ethyl}morpholine hydrochloride or solvates or a prodrug thereof. Preferably, the compound is 4-{2-[5-Methyl-1-(naphthalen-2-yl)-1H-pyrazol-3-yloxy]ethyl}morpholine hydrochloride.

In another preferred embodiment of the use as defined above the compound is a compound according to formula II:

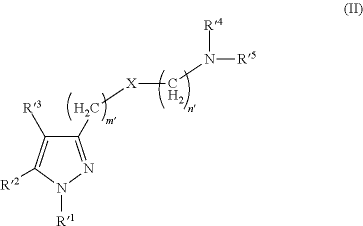

wherein
R′¹ represents substituted or unsubstituted aromatic or non-aromatic heterocyclyl; substituted or unsubstituted aryl; or substituted or unsubstituted cycloalkyl;
R′² and R′³, identical or different, represent a hydrogen atom; F; Cl; Br; I; CF₃; OH; SH; NH₂; CN; substituted or unsubstituted alkyl; substituted or unsubstituted alkenyl; substituted or unsubstituted alkoxy; substituted or unsubstituted cycloalkyl; substituted or unsubstituted aryl; substituted or unsubstituted, aromatic or non-aromatic heterocyclyl; substituted or unsubstituted cycloalkylalkyl; substituted or unsubstituted arylalkyl; substituted or unsubstituted, aromatic or non-aromatic heterocyclylalkyl; a (C═O)—R′⁷ group; a (C═O)—O—R′⁸ group; a S(O)ₜ—R′⁹ group; or a (C═O)—NR′¹⁰R′¹¹ group;
R′⁴ and R′⁵, identical or different, represent a hydrogen atom; substituted or unsubstituted alkyl; substituted or unsubstituted alkenyl; substituted or unsubstituted alkoxy; substituted or unsubstituted cycloalkyl; substituted or unsubstituted aryl; substituted or unsubstituted, aromatic or non-aromatic heterocyclyl; substituted or unsubstituted cycloalkylalkyl; substituted or unsubstituted arylalkyl; substituted or unsubstituted, aromatic or non-aromatic heterocyclylalkyl; a (C═O)—R′⁷ group; a (C═O)—O—R′⁸ group; a S(O)ₜ—R′⁹ group; or a (C═O)—NR′¹⁰R′¹¹ group;
or
together form, with the nitrogen atom to which they are attached, a substituted or unsubstituted, aromatic or non-aromatic heterocyclyl group;
X represents an oxygen atom or a CH—R′¹² group wherein R′¹² is selected from H, CH₃, SH, OH, NH₂, CF₃, Cl, F, Br, I, and CN;

m′ is selected from 1, 2, 3 and 4;

n′ is selected from 1, 2, 3 and 4;

t′ is selected from 1, 2 and 3;

R′⁷, R′⁸, R′⁹, R′¹⁰ and R′¹¹, identical or different, represent a hydrogen atom; substituted or unsubstituted C₁₋₆ alkyl; substituted or unsubstituted C₁₋₆ alkenyl; substituted or unsubstituted C₁₋₆ alkoxy; substituted or unsubstituted cycloalkyl; substituted or unsubstituted aryl; substituted or unsubstituted, aromatic or non-aromatic heterocyclyl; substituted or unsubstituted cycloalkylalkyl; substituted or unsubstituted arylalkyl; substituted or unsubstituted, aromatic or non-aromatic heterocyclylalkyl;

or a pharmaceutically acceptable salt, isomer, prodrug or solvate thereof, or preferably a pharmaceutically acceptable salt or isomer thereof.

In one embodiment of the use as defined above the compound is characterized in that R′¹ in formula (II) above is selected from a 5- to 10 membered substituted or unsubstituted, aromatic or non-aromatic heterocyclyl group which preferably contains N, O or S as ring member; a 5- to 10 membered substituted or unsubstituted aryl group; and a 5- to 10 membered substituted or unsubstituted cycloalkyl group.

In a preferred embodiment of the use as defined above the compound is characterized in that R′¹ in formula (II) above is selected from substituted or unsubstituted cyclopentyl, substituted or unsubstituted cyclohexyl, substituted or unsubstituted phenyl, substituted or unsubstituted naphtyl, substituted or unsubstituted thiophene, substituted or unsubstituted benzothiophene, substituted or unsubstituted benzofuran, substituted or unsubstituted pyridine and substituted or unsubstituted quinoline.

In a still more preferred embodiment of the use as defined above the compound is characterized in that R′¹ in formula (II) above is selected from the group consisting of: 2-thienyl, 3-thienyl, 2,5-dichloro-3-thienyl, 2,3-dichloro-5-thienyl, 2,3-dichloro-4-thienyl, 2-benzothienyl, 3-benzothienyl, 4-benzothienyl, 5-benzothienyl, 7-benzothienyl, 2-benzofuryl, 5-benzofuryl, 2-pyridyl, 3-pyridyl, 4-pyridyl, 2-quinolyl, 3-quinolyl, 5-quinolyl, 6-quinolyl and 3,4-dichlorophenyl.

In another preferred embodiment of the use as defined above the compound is characterized in that R′¹ in formula (II) above is an α or β naphthyl, preferably selected from the following α or β naphthyl groups: 7-hydroxy-2-naphtyl, 6-hydroxy-2-naphtyl, 5-hydroxy-2-naphtyl, 6-fluoro-2-naphtyl, 6-methoxy-2-naphtyl, 6-bromo-2-naphtyl, 6-hydroxymethyl-2-naphtyl, 6-fluromethyl-2-naphtyl, 7-hydroxy-1-naphtyl, 6-hydroxy-1-naphtyl, 5-hydroxy-1-naphtyl, 5-fluoro-1-naphtyl, 5-bromo-1-naphtyl and 1-naphtyl.

In another embodiment of the use as defined above the compound is characterized in that R′² and R′³ in formula (II) are independently selected from H and substituted or unsubstituted C₁₋₆ alkyl group, preferably methyl. More particular embodiments of the use as defined above are those wherein R′² is methyl and R′³ is H, or R′² and R′³ are simultaneously H, or simultaneously methyl.

In a preferred embodiment of the use as defined above the compound is characterized in that R′⁴ and R′⁵ in formula (II) form together with the nitrogen atom to which they are attached a substituted or unsubstituted heterocyclyl group. More preferably, R′⁴ and R′⁵ form together a morpholine-4-yl group, a piperidine-4-yl group, pyrrolidine-4-yl group or a piperazine-4-yl group.

Preferred values for m′ and n′ in formula (II) are independently 1 and 2.

Further, X preferably represents an oxygen atom or a —CH₂— group.

In a preferred variant of the invention the sigma ligand of general formula (I) is selected from:
[65] 4-(2-((1-(3,4-dichlorophenyl)-5-methyl-1H-pyrazol-3-yl)methoxy)ethyl)morpholine,
[66] 4-(2-((5-methyl-1-(naphthalen-2-yl)-1H-pyrazol-3-yl)methoxy)ethyl)morpholine,
[67] 4-(3-(1-(3,4-dichlorophenyl)-5-methyl-1H-pyrazol-3-yl)propyl)morpholine
[68] 4-(3-(5-methyl-1-(naphthalen-2-yl)-1H-pyrazol-3-yl)propyl)morpholine
[69] 4-(2-(2-(1-(3,4-dichlorophenyl)-5-methyl-1H-pyrazol-3-yl)ethoxy)ethyl)morpholine
[70] 4-(2-((1-cyclohexyl-5-methyl-1H-pyrazol-3-yl)methoxy)ethyl)morpholine
[71] 4-(3-(1-cyclohexyl-5-methyl-1H-pyrazol-3-yl)propyl)morpholine
[72] 1-(3,4-dichlorophenyl)-5-methyl-3-((2-(pyrrolidin-1-yl)ethoxy)methyl)-1H-pyrazole
[73] 1-(2-((1-(3,4-dichlorophenyl)-5-methyl-1H-pyrazol-3-yl)methoxy)ethyl)piperidine
[74] 1-(4-(2-((1-(3,4-dichlorophenyl)-5-methyl-1H-pyrazol-3-yl)methoxy)ethyl)piperazin-1-yl)ethanone
[75] (2S,6R)-4-(2-((1-(3,4-dichlorophenyl)-5-methyl-1H-pyrazol-3-yl)methoxy)ethyl)-2,6-dimethylmorpholine
[76] 4-(2-((5-methyl-1-(quinolin-3-yl)-1H-pyrazol-3-yl)methoxy)ethyl)morpholine
[77] 4-(4-(1-(3,4-dichlorophenyl)-5-methyl-1H-pyrazol-3-yl)butyl)morpholine
[78] 4-(3-(5-methyl-1-(quinolin-3-yl)-1H-pyrazol-3-yl)propyl)morpholine
[79] 4-(2-((1-(3,4-dichlorophenyl)-1H-pyrazol-3-yl)methoxy)ethyl)morpholine
[80] 4-(2-((1-(3,4-dichlorophenyl)-4,5-dimethyl-1H-pyrazol-3-yl)methoxy)ethyl)morpholine
[81] 4-(3-(1-(quinolin-3-yl)-1H-pyrazol-3-yl)propyl)morpholine
[82] 4-(4-(1-(3,4-dichlorophenyl)-1H-pyrazol-3-yl)butyl)morpholine
[83] 4-(4-(5-methyl-1-(quinolin-3-yl)-1H-pyrazol-3-yl)butyl)morpholine
[84] 4-(3-((1-cyclohexyl-5-methyl-1H-pyrazol-3-yl)methoxy)propyl)morpholine
[85] 4-(2-((1-cyclopentyl-5-methyl-1H-pyrazol-3-yl)methoxy)ethyl)morpholine
[86] 1-(4-(2-((1-cyclohexyl-5-methyl-1H-pyrazol-3-yl)methoxy)ethyl)piperazin-1-yl)ethanone hydrochloride
[87] (3S,5R)-1-(2-((1-cyclohexyl-5-methyl-1H-pyrazol-3-yl)methoxy)ethyl)-3,5-dimethyl piperazine hydrochloride
[88] 4-(2-(2-(1-cyclohexyl-5-methyl-1H-pyrazol-3-yl)ethoxy)ethyl)morpholine hydrochloride
[89] 4-(2-((1-cyclohexyl-1H-pyrazol-3-yl)methoxy)ethyl)morpholine hydrochloride
[90] 4-(2-((1-cyclohexyl-4,5-dimethyl-1H-pyrazol-3-yl)methoxy)ethyl)morpholine hydrochloride
[91] 1-(4-(2-((1-cyclohexyl-1H-pyrazol-3-yl)methoxy)ethyl)piperazin-1-yl)ethanone
[92] 1-(4-(3-((1-cyclohexyl-1H-pyrazol-3-yl)methoxy)propyl)piperazin-1-yl)ethanone
[94] 1-(4-(4-((1-cyclohexyl-1H-pyrazol-3-yl)methoxy)butyl)piperazin-1-yl)ethanone
[95] 1-(4-(4-((1-cyclohexyl-5-methyl-1H-pyrazol-3-yl)methoxy)butyl)piperazin-1-yl)ethanone
[96] 1-(4-(3-((1-cyclohexyl-5-methyl-1H-pyrazol-3-yl)methoxy)propyl)piperazin-1-yl)ethanone
[97] 1-(4-(2-((1-(3,4-dichlorophenyl)-1H-pyrazol-3-yl)methoxy)ethyl)piperazin-1-yl)ethanone
[98] 1-(4-(3-((1-(3,4-dichlorophenyl)-1H-pyrazol-3-yl)methoxy)propyl)piperazin-1-yl)ethanone
[99] 1-(4-(4-((1-(3,4-dichlorophenyl)-1H-pyrazol-3-yl)methoxy)butyl)piperazin-1-yl)ethanone
1-(4-(3-((1-(3,4-dichlorophenyl)-5-methyl-1H-pyrazol-3-yl)methoxy)propyl) piperazin-1-yl)ethanone
1-(4-(3-((1-(3,4-difluorophenyl)-1H-pyrazol-3-yl)methoxy)propyl)piperazin-1-yl)ethanone
1-(4-(3-((1-(3,4-difluorophenyl)-5-methyl-1H-pyrazol-3-yl)methoxy)propyl)piperazin-1-yl)ethanone
1-(4-(2-((1-(3,4-difluorophenyl)-5-methyl-1H-pyrazol-3-yl)methoxy)ethyl)piperazin-1-yl)ethanone
1-(4-(2-((1-(3,4-difluorophenyl)-1H-pyrazol-3-yl)methoxy)ethyl)piperazin-1-yl)ethanone
4-(2-((1-(3,4-difluorophenyl)-5-methyl-1H-pyrazol-3-yl)methoxy)ethyl)morpholine
4-(2-((1-(3,4-difluorophenyl)-1H-pyrazol-3-yl)methoxy)ethyl)morpholine
4-(3-((1-(3,4-difluorophenyl)-5-methyl-1H-pyrazol-3-yl)methoxy)propyl)morpholine
1-(4-(2-((1-cyclohexyl-5-methyl-1H-pyrazol-3-yl)methoxy)ethyl)piperazin-1-yl)propan-1-one
1-(4-(2-((1-cyclohexyl-5-methyl-1H-pyrazol-3-yl)methoxy)ethyl)piperazin-1-yl)-2-methylpropan-1-one
1-(4-(2-((1-cyclohexyl-1H-pyrazol-3-yl)methoxy)ethyl)piperazin-1-yl)propan-1-one
1-(4-(2-((1-cyclohexyl-1H-pyrazol-3-yl)methoxy)ethyl)piperazin-1-yl)-2-methyl propan-1-one
1-(4-(2-((1-(3,4-dichlorophenyl)-5-methyl-1H-pyrazol-3-yl)methoxy)ethyl) piperazin-1-yl)propan-1-one
1-(4-(2-((1-(3,4-dichlorophenyl)-5-methyl-1H-pyrazol-3-yl)methoxy)ethyl) piperazin-1-yl)-2-methylpropan-1-one
1-(4-(2-((1-(3,4-dichlorophenyl)-1H-pyrazol-3-yl)methoxy)ethyl)piperazin-1-yl) propan-1-one
1-(4-(2-((1-(3,4-dichlorophenyl)-1H-pyrazol-3-yl)methoxy)ethyl)piperazin-1-yl)-2-methylpropan-1-one
   or a pharmaceutically acceptable salt, prodrug or solvate thereof, preferably a pharmaceutically acceptable salt thereof.

In a more preferred embodiment of the use as defined above the compound is 1-(4-(2-((1-(3,4-difluorophenyl)-1H-pyrazol-3-yl)methoxy)ethyl)piperazin-1-yl)ethanone or its pharmaceutically acceptable salts, solvates or a prodrug thereof. Preferably the compound is 1-(4-(2-((1-(3,4-difluorophenyl)-1H-pyrazol-3-yl)methoxy)ethyl)piperazin-1-yl)ethanone or its pharmaceutically acceptable salts.

In a more preferred embodiment of the use as defined above the compound is selected from 1-(4-(2-((1-(3,4-difluorophenyl)-1H-pyrazol-3-yl) methoxy)ethyl)piperazin-1-yl)ethanone hydrochloride or its pharmaceutically acceptable solvates or a prodrug thereof. Preferably, the compound is 1-(4-(2-((1-(3,4-difluorophenyl)-1H-pyrazol-3-yl) methoxy)ethyl)piperazin-1-yl)ethanone hydrochloride.

In a most preferred embodiment of the use as defined above the compound is selected from 1-(4-(2-((1-(3,4-difluorophenyl)-1H-pyrazol-3-yl)methoxy)ethyl)piperazin-1-yl)ethanone or its pharmaceutically acceptable salts or solvates, and 1-(4-(2-((1-(3,4-difluorophenyl)-1H-pyrazol-3-yl) methoxy)ethyl)piperazin-1-yl)ethanone hydrochloride or solvates thereof, or

the compound is selected from 4-{2-[5-Methyl-1-(naphthalen-2-yl)-1H-pyrazol-3-yloxy]ethyl}morpholine or its pharmaceutically acceptable salts or solvates and 4-{2-[5-Methyl-1-(naphthalen-2-yl)-1H-pyrazol-3-yloxy]ethyl}morpholine hydrochloride or solvates thereof.

In a most preferred embodiment of the use as defined above the compound is selected from 1-(4-(2-((1-(3,4-difluorophenyl)-1H-pyrazol-3-yl)methoxy)ethyl)piperazin-1-yl)ethanone or its pharmaceutically acceptable salts, and 1-(4-(2-((1-(3,4-difluorophenyl)-1H-pyrazol-3-yl) methoxy)ethyl)piperazin-1-yl)ethanone hydrochloride, or

the compound is selected from 4-{2-[5-Methyl-1-(naphthalen-2-yl)-1H-pyrazol-3-yloxy]ethyl}morpholine or its pharmaceutically acceptable salts and 4-{2-[5-Methyl-1-(naphthalen-2-yl)-1H-pyrazol-3-yloxy]ethyl}morpholine hydrochloride.

“Alkyl” refers to a straight or branched hydrocarbon chain radical consisting of carbon and hydrogen atoms, containing no saturation, having one to eight carbon atoms, and which is attached to the rest of the molecule by a single bond, e.g., methyl, ethyl, n-propyl, i-propyl, n-butyl, t-butyl, n-pentyl, etc. Alkyl radicals may be optionally substituted by one or more substituents such as a aryl, halo, hydroxy, alkoxy, carboxy, cyano, carbonyl, acyl, alkoxycarbonyl, amino, nitro, mercapto, alkylthio, etc. If substituted by aryl we have an “Aralkyl” radical, such as benzyl and phenethyl.

“Alkenyl” refers to an alkyl radical having at least 2 C atoms and having one or more unsaturated bonds.

“Cycloalkyl” refers to a stable 3- to 10-membered monocyclic or bicyclic radical which is saturated or partially saturated, and which consist solely of carbon and hydrogen atoms, such as cyclohexyl or adamantyl. Unless otherwise stated specifically in the specification, the term“cycloalkyl” is meant to include cycloalkyl radicals which are optionally substituted by one or more substituents such as alkyl, halo, hydroxy, amino, cyano, nitro, alkoxy, carboxy, alkoxycarbonyl, etc.

“Aryl” refers to single and multiple ring radicals, including multiple ring radicals that contain separate and/or fused aryl groups. Typical aryl groups contain from 1 to 3 separated or fused rings and from 6 to about 18 carbon ring atoms, such as phenyl, naphthyl, indenyl, fenanthryl or anthracyl radical. The aryl radical may be optionally substituted by one or more substituents such as hydroxy, mercapto, halo, alkyl, phenyl, alkoxy, haloalkyl, nitro, cyano, dialkylamino, aminoalkyl, acyl, alkoxycarbonyl, etc.

“Heterocyclyl” refers to a stable 3- to 15 membered ring radical which consists of carbon atoms and from one to five heteroatoms selected from the group consisting of nitrogen, oxygen, and sulfur, preferably a 4- to 8-membered ring with one or more heteroatoms, more preferably a 5- or 6-membered ring with one or more heteroatoms. It may be aromatic or not aromatic. For the purposes of this invention, the heterocycle may be a monocyclic, bicyclic or tricyclic ring system, which may include fused ring systems; and the nitrogen, carbon or sulfur atoms in the heterocyclyl radical may be optionally oxidised; the nitrogen atom may be optionally quaternized; and the heterocyclyl radical may be partially or fully saturated or aromatic. Examples of such heterocycles include, but are not limited to, azepines, benzimidazole, benzothiazole, furan, isothiazole, imidazole, indole, piperidine, piperazine, purine, quinoline, thiadiazole, tetrahydrofuran, coumarine, morpholine; pyrrole, pyrazole, oxazole, isoxazole, triazole, imidazole, etc.

“Alkoxy” refers to a radical of the formula —ORa where Ra is an alkyl radical as defined above, e.g., methoxy, ethoxy, propoxy, etc.

“Amino” refers to a radical of the formula-NH₂, —NHRa or —NRaRb, optionally quaternized, wherein Ra and Rb is an alkyl radical as defined above, e.g., methoxy, ethoxy, propoxy, etc.

“Halo” or “hal” refers to bromo, chloro, iodo or fluoro.

References herein to substituted groups in the compounds of the present invention refer to the specified moiety that may be substituted at one or more available positions by one or more suitable groups, e.g., halogen such as fluoro, chloro, bromo and iodo; cyano; hydroxyl; nitro; azido; alkanoyl such as a C₁₋₆ alkanoyl group such as acyl and the like; carboxamido; alkyl groups including those groups having 1 to about 12 carbon atoms or from 1 to about 6 carbon atoms and more preferably 1-3 carbon atoms; alkenyl and alkynyl groups including groups having one or more unsaturated linkages and from 2 to about 12 carbon or from 2 to about 6 carbon atoms; alkoxy groups having one or more oxygen linkages and from 1 to about 12 carbon atoms or 1 to about 6 carbon atoms; aryloxy such as phenoxy; alkylthio groups including those moieties having one or more thioether linkages and from 1 to about 12 carbon atoms or from 1 to about 6 carbon atoms; alkylsulfinyl groups including those moieties having one or more sulfinyl linkages and from 1 to about 12 carbon atoms or from 1 to about 6 carbon atoms; alkylsulfonyl groups including those moieties having one or more sulfonyl linkages and from 1 to about 12 carbon atoms or from 1 to about 6 carbon atoms; aminoalkyl groups such as groups having one or more N atoms and from 1 to about 12 carbon atoms or from 1 to about 6 carbon atoms; carbocylic aryl having 6 or more carbons, particularly phenyl or naphthyl and aralkyl such as benzyl. Unless otherwise indicated, an optionally substituted group may have a substituent at each substitutable position of the group, and each substitution is independent of the other.

Unless otherwise stated, the compounds of the invention are also meant to include compounds which differ only in the presence of one or more isotopically enriched atoms. For example, compounds having the present structures except for the replacement of a hydrogen by a deuterium or tritium, or the replacement of a carbon by a ¹³C- or ¹⁴C-enriched carbon or ¹⁵N-enriched nitrogen are within the scope of this invention.

The term “pharmaceutically acceptable salts, solvates, prodrugs” refers to any pharmaceutically acceptable salt, ester, solvate, or any other compound which, upon administration to the recipient is capable of providing (directly or indirectly) a compound as described herein. However, it will be appreciated that non-pharmaceutically acceptable salts also fall within the scope of the invention since those may be useful in the preparation of pharmaceutically acceptable salts. The preparation of salts, prodrugs and derivatives can be carried out by methods known in the art.

For instance, pharmaceutically acceptable salts of compounds provided herein are synthesized from the parent compound which contains a basic or acidic moiety by conventional chemical methods. Generally, such salts are, for example, prepared by reacting the free acid or base forms of these compounds with a stoichiometric amount of the appropriate base or acid in water or in an organic solvent or in a mixture of the two. Generally, nonaqueous media like ether, ethyl acetate, ethanol, isopropanol or acetonitrile are preferred. Examples of the acid addition salts include mineral acid addition salts such as, for example, hydrochloride, hydrobromide, hydroiodide, sulphate, nitrate, phosphate, and organic acid addition salts such as, for example, acetate, maleate, fumarate, citrate, oxalate, succinate, tartrate, malate, mandelate, methanesulphonate and p-toluenesulphonate. Examples of the alkali addition salts include inorganic salts such as, for example, sodium, potassium, calcium, ammonium, magnesium, aluminium and lithium salts, and organic alkali salts such as, for example, ethylenediamine, ethanolamine, N,N-dialkylenethanolamine, triethanolamine, glucamine and basic aminoacids salts.

Particularly favored derivatives or prodrugs are those that increase the bioavailability of the compounds of this invention when such compounds are administered to a patient (e.g., by allowing an orally administered compound to be more readily absorbed into the blood) or which enhance delivery of the parent compound to a biological compartment (e.g., the brain or lymphatic system) relative to the parent species.

Any compound that is a prodrug of a compound of formula (I) is within the scope of the invention. The term “prodrug” is used in its broadest sense and encompasses those derivatives that are converted in vivo to the compounds of the invention. Such derivatives would readily occur to those skilled in the art, and include, depending on the functional groups present in the molecule and without limitation, the following derivatives of the present compounds: esters, amino acid esters, phosphate esters, metal salts sulfonate esters, carbamates, and amides. Examples of well-known methods of producing a prodrug of a given acting compound are known to those skilled in the art and can be found e.g. in Krogsgaard-Larsen et al. “Textbook of Drug design and Discovery” Taylor & Francis (April 2002).

The compounds of the invention may be in crystalline form either as free compounds or as solvates and it is intended that both forms are within the scope of the present invention. Methods of solvation are generally known within the art. Suitable solvates are pharmaceutically acceptable solvates. In a particular embodiment the solvate is a hydrate.

The compounds of formula (I) and formula (II) or their salts or solvates are preferably in pharmaceutically acceptable or substantially pure form. By pharmaceutically acceptable form is meant, inter alia, having a pharmaceutically acceptable level of purity excluding normal pharmaceutical additives such as diluents and carriers, and including no material considered toxic at normal dosage levels. Purity levels for the drug substance are preferably above 50%, more preferably above 70%, most preferably above 90%. In a preferred embodiment it is above 95% of the compound of formula (I), or of its salts, solvates or prodrugs.

The compounds of the present invention represented by the above described formula (I) and formula (II) may include enantiomers depending on the presence of chiral centres or isomers depending on the presence of multiple bonds (e.g. Z, E). The single isomers, enantiomers or diastereoisomers and mixtures thereof fall within the scope of the present invention.

The compounds of formula (I) and their salts or solvates can be prepared as disclosed in the previous application WO2006/021462.

The compounds of formula (II) and their salts or solvates can be prepared as disclosed in the previous application WO 2011/147910.

The obtained reaction products may, if desired, be purified by conventional methods, such as crystallisation and chromatography. Where the above described processes for the preparation of compounds of the invention give rise to mixtures of stereoisomers, these isomers may be separated by conventional techniques such as preparative chromatography. If there are chiral centers the compounds may be prepared in racemic form, or individual enantiomers may be prepared either by enantiospecific synthesis or by resolution.

One preferred pharmaceutically acceptable form is the crystalline form, including such form in pharmaceutical composition. In the case of salts and solvates the additional ionic and solvent moieties must also be non-toxic. The compounds of the invention may present different polymorphic forms, it is intended that the invention encompasses all such forms.

Another aspect of this invention relates to a method of treating or preventing osteoarthritis, and/or to a method of treating or preventing pain due to osteoarthritis, which method(s) comprises administering to a patient in need of such a treatment a therapeutically effective amount of a compound as above defined or a pharmaceutical composition thereof.

A preferred embodiment relates to the method of treating or preventing osteoarthritis, which method comprises administering to a patient in need of such a treatment a therapeutically effective amount of a compound as above defined or a pharmaceutical composition thereof.

A preferred embodiment relates to the method of treating osteoarthritis, which method comprises administering to a patient in need of such a treatment a therapeutically effective amount of a compound as above defined or a pharmaceutical composition thereof.

A preferred embodiment relates to the method of preventing osteoarthritis, which method comprises administering to a patient in need of such a treatment a therapeutically effective amount of a compound as above defined or a pharmaceutical composition thereof.

Another aspect of this invention relates to a method of treating or preventing pain due to osteoarthritis, which method comprises administering to a patient in need of such a treatment a therapeutically effective amount of a compound as above defined or a pharmaceutical composition thereof.

Another aspect of this invention relates to a method of treating pain due to osteoarthritis, which method comprises administering to a patient in need of such a treatment a therapeutically effective amount of a compound as above defined or a pharmaceutical composition thereof.

Another aspect of this invention relates to a method of preventing pain due to osteoarthritis, which method comprises administering to a patient in need of such a treatment a therapeutically effective amount of a compound as above defined or a pharmaceutical composition thereof.

In another aspect the invention is directed to a use of the compounds as above defined in the preparation of a medicament for the treatment or prevention of osteoarthritis, and/or for the treatment or prevention of pain due to osteoarthritis.

A preferred embodiment relates to a use of the compounds as above defined in the preparation of a medicament for the treatment of osteoarthritis.

A preferred embodiment relates to a use of the compounds as above defined in the preparation of a medicament for the prevention of osteoarthritis.

A preferred embodiment relates to a use of the compounds as above defined in the preparation of a medicament for the treatment or prevention of pain due to osteoarthritis.

A preferred embodiment relates to a use of the compounds as above defined in the preparation of a medicament for the treatment of pain due to osteoarthritis.

A preferred embodiment relates to a use of the compounds as above defined in the preparation of a medicament for the prevention of pain due to osteoarthritis.

The present invention further provides pharmaceutical compositions comprising a compound of this invention, or a pharmaceutically acceptable salt, derivative, prodrug or stereoisomers thereof together with a pharmaceutically acceptable carrier, adjuvant, or vehicle, for administration to a patient.

In another aspect the invention is thus directed to a pharmaceutical composition comprising a compound as defined above, wherein the composition further comprises a pharmaceutically acceptable carrier, adjuvant and/or vehicle.

Examples of pharmaceutical compositions include any solid (tablets, pills, capsules, granules etc.) or liquid (solutions, suspensions or emulsions) composition for oral, topical or parenteral administration.

In a preferred embodiment the pharmaceutical compositions are in oral form, either solid or liquid. Suitable dose forms for oral administration may be tablets, capsules, syrops or solutions and may contain conventional excipients known in the art such as binding agents, for example syrup, acacia, gelatin, sorbitol, tragacanth, or polyvinylpyrrolidone; fillers, for example lactose, sugar, maize starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulfate.

The solid oral compositions may be prepared by conventional methods of blending, filling or tabletting. Repeated blending operations may be used to distribute the active agent throughout those compositions employing large quantities of fillers. Such operations are conventional in the art. The tablets may for example be prepared by wet or dry granulation and optionally coated according to methods well known in normal pharmaceutical practice, in particular with an enteric coating.

The pharmaceutical compositions may also be adapted for parenteral administration, such as sterile solutions, suspensions or lyophilized products in the appropriate unit dosage form. Adequate excipients can be used, such as bulking agents, buffering agents or surfactants.

The mentioned formulations will be prepared using standard methods such as those described or referred to in the Spanish and US Pharmacopoeias and similar reference texts. Administration of the compounds or compositions of the present invention may be by any suitable method, such as intravenous infusion, oral preparations, and intraperitoneal and intravenous administration. Oral administration is preferred because of the convenience for the patient and the chronic character of the diseases to be treated.

Generally an effective administered amount of a compound of the invention will depend on the relative efficacy of the compound chosen, the severity of the disorder being treated and the weight of the sufferer. However, active compounds will typically be administered once or more times a day for example 1, 2, 3 or 4 times daily, with typical total daily doses in the range of from 0.1 to 1000 mg/kg/day.

Importantly, as shown below, the compounds of the present invention display a long-term pain-improving effect and a long-term mobility-improving effect on test animals, most likely through preventing, inhibiting and/or interfering with cartilage degeneration (and subchondral bone degeneration). Thus the compounds of the invention act as “Disease modifying Osteoarthritis Drugs” (DMOAD s).

Due to the long-term, accumulative effect it is possible to also administer the compounds of the invention in several desirable dosage regimen.

In a preferred embodiment of the use of the compounds of the invention the compounds, optionally in the form of a pharmaceutical composition, are administered once daily.

In a preferred embodiment of the use of the compounds of the invention the compounds, optionally in the form of a pharmaceutical composition, are administered once every 36 hours.

In a preferred embodiment of the use of the compounds of the invention the compounds, optionally in the form of a pharmaceutical composition, are administered once every 2 days.

In another preferred embodiment of the use of the compounds of the invention the compounds, optionally in the form of a pharmaceutical composition, are administered once every 3 days.

In a preferred embodiment of the use of the compounds of the invention the compounds, optionally in the form of a pharmaceutical composition, are administered once every 4 days.

In a preferred embodiment of the use of the compounds of the invention the compounds, optionally in the form of a pharmaceutical composition, are administered once every 5 days.

In a preferred embodiment of the use of the compounds of the invention the compounds, optionally in the form of a pharmaceutical composition, are administered once every 6 days.

In a preferred embodiment of the use of the compounds of the invention the compounds, optionally in the form of a pharmaceutical composition, are administered once every 7 days.

The compounds and compositions of this invention may be used with other drugs to provide a combination therapy. The other drugs may form part of the same composition, or be provided as a separate composition for administration at the same time or at different time.

The following examples are given only as further illustration of the invention, they should not be taken as a definition of the limits of the invention.

### EXAMPLES

### Example 1

### Synthesis of 4-{2-[5-Methyl-1-(naphthalen-2-yl)-1H-pyrazol-3-yloxy]ethyl}morpholine (compound 63) and its hydrochloride salt

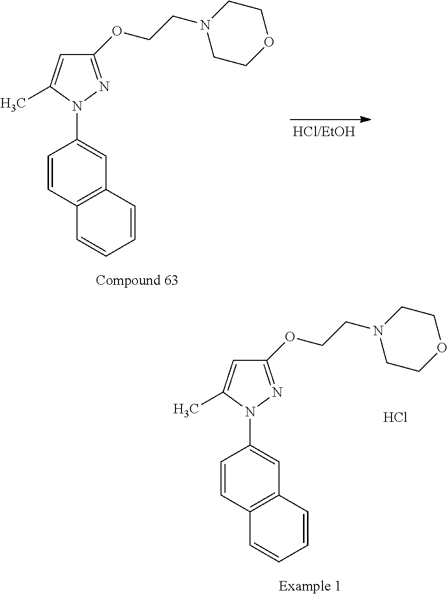 [SMILES: Cc1cc(OCCN2CCOCC2)nn1-c1ccc2ccccc2c1 ; Cc1cc(OCCN2CCOCC2)nn1-c1ccc2ccccc2c1 ; Cl]

Compound 63 can be prepared as disclosed in the previous application WO2006/021462 (Compound 63 is example 61 in WO2006/021462). Its hydrochloride can be obtained according the following procedure:

Compound 63 (6.39 g) was dissolved in ethanol saturated with HCl, the mixture was stirred then for some minutes and evaporated to dryness. The residue was crystallized from isopropanol. The mother liquors from the first crystallization afforded a second crystallization by concentrating. Both crystallizations taken together yielded 5.24 g (63%) of the corresponding hydrochloride salt (m.p.=197-199° C.).

1H-NMR (DMSO-d6) δ ppm: 10.85 (bs, 1H), 7.95 (m, 4H), 7.7 (dd, J=2, 2, 8.8 Hz, 1H), 7.55 (m, 2H), 5.9 (s, 1H), 4.55 (m, 2H), 3.95 (m, 2H), 3.75 (m, 2H), 3.55-3.4 (m, 4H), 3.2 (m, 2H), 2.35 (s, 3H).

HPLC purity: 99.8%.

### Example 2

### Synthesis of 1-(4-(2-((1-(3,4-difluorophenyl)-1H-pyrazol-3-yl)methoxy)ethyl)piperazin-1-yl)ethanone

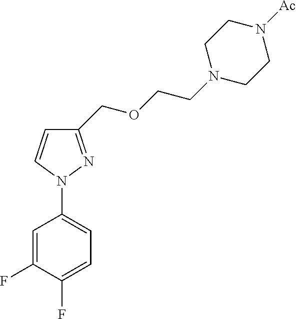 [SMILES: CC(=O)N1CCN(CCOCc2ccn(-c3ccc(F)c(F)c3)n2)CC1]

Example 2 can be can be prepared as disclosed in the previous application WO2011/147910 (Example 2 is example 39 in WO2011/147910).

### Example 3: Synthesis of 1-(4-(2-((1-(3,4-difluorophenyl)-1H-pyrazol-3-yl)methoxy)ethyl)piperazin-1-yl)ethanone hydrochloride

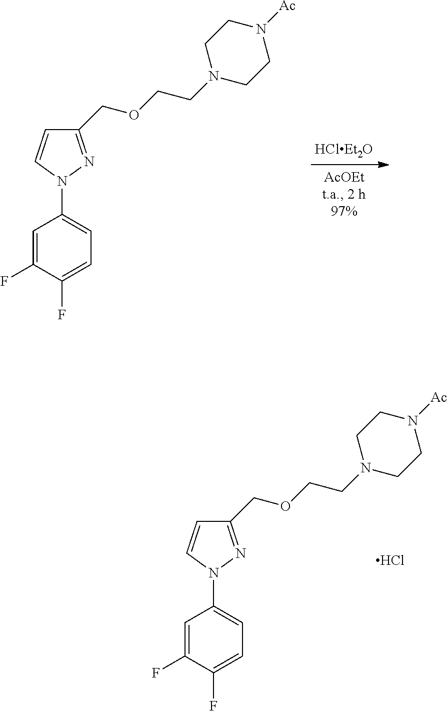 [SMILES: CC(=O)N1CCN(CCOCc2ccn(-c3ccc(F)c(F)c3)n2)CC1 ; CC(=O)N1CCN(CCOCc2ccn(-c3ccc(F)c(F)c3)n2)CC1 ; Cl]

To a solution of 1-(4-(2-((1-(3,4-difluorophenyl)-1H-pyrazol-3-yl) methoxy)ethyl)piperazin-1-yl)ethanone (57.41 g, 157.55 mmol) in ethyl acetate (900 mL), HCl.Et₂O (2.0 M, 86.7 mL, 173.30 mmol) was added and the mixture was stirred at room temperature for 2 h. The mixture was evaporated to dryness, ethyl ether (300 mL) was added and evaporated again. This process was repeated two times with CH₂Cl₂ and ethyl ether. The solid thus obtained was triturated with hexane (400 mL) and filtered, washed with hexane (200 mL) and with ethyl ether/hexane (1:1, 100 mL). The solid was dried to give the title compound (61.2 g, 97% yield).

RMN-¹H (CD₃OD, 400 MHz, 8): 8.24 (d, J=2.7 Hz, 1H, ArH); 7.76 (ddd, J=11.7, 7.0, 2.7 Hz, 1H, ArH); 7.61-7.55 (m, 1H, ArH); 7.47-7.37 (m, 1H, ArH); 6.58 (d, J=2.5 Hz, 1H, ArH); 4.71 (s, 2H, CH₂); 4.59 (sa, 1H, CH₂); 4.20-4.05 (m, 1H, CH₂); 3.96-3.85 (m, 2H, CH₂); 3.69-3.39 (m, 4H, CH₂); 3.24-2.99 (m, 2H, CH₂); 2.14 (s, 3H, CH₃). (FIG. 4)

EM-ESI+m/z: 365 (M+1-HCl).

### Pharmacological Data

### Animals

Male Sprague-Dawley rats (Harlan, San Pietro Nastisone, Udine, Italia), weighing 50-75 g on arrival at the laboratory, were used. Animals were allowed to acclimate for one week in groups of five per cage in a room with constant temperature (21±1° C.) and relative humidity (60%), and free access to food and water ad libitum. Automatic light-on and -off allowed alternate light-dark cycles of 12 h:12 h, with light-on at 7:00 a.m. Procedures were approved by the Committee on Animal Research Ethics of ESTEVE and conformed to the guidelines of the IASP (Zimmermann, 1983). In particular, the duration of the experiments was kept as short as possible and the number of rats used was minimized.

Reference: Zimmermann M. (1983). Ethical guidelines for investigations of experimental pain in conscious animals. Pain, 16, 109-10.

### MIA-Induced Osteoarthritis

Osteoarthritis in rats was induced by intraarticular (i.a.) injection of monosodium iodoacetate (MIA) in the right knee joint, essentially as described by Dunham et al. (1993). After one week of acclimation, rats were briefly anesthetized with isoflurane (5% in 02 at 600 cc/min; IsoFlo®, Veterinaria ESTEVE, MI, Italy) until lack of response to a toe pinch. Surgical area was swabbed with chlorohexidine and alcohol, then a single injection of MIA (50 μl of a 40 mg/ml solution in 0.9% saline, i.e. equal to 2 mg/injection) was delivered using a 28-gauge needle inserted into the joint space of the knee through the intra-patellar ligament, by a gentle flexion of the knee. Sham animals received, in parallel, an equal volume of 0.9% sterile saline.

The MIA dose to obtain a prolonged, marked decrease in mechanical thresholds to von Frey filaments pressure was selected on the basis of a dose-response experiment (not shown). After recovery, animals were returned to their home cage.

### Nociceptive Testings

Rats not acclimated to the test conditions beforehand were assigned into wire mesh bottom cylinders (transparent metacrylate, 300 mm high×200 mm diameter) and allowed to acclimatize prior the start of the experiment. Tactile allodynia was assessed by determination of the paw withdrawal threshold (PWT) to von Frey filaments stimulation, starting 1 to 15 grams, on the plantar surface of the hind paw. Each filament was applied 3 s until a withdrawal response occurred. A single response indicated a positive response. PWTs were assessed in both the inflamed (ipsilateral) and non-inflamed (contralateral) hind paw and expressed as grams. Control thresholds in the contralateral paw were established in a similar fashion. Nociceptive thresholds were measured prior to MIA injection (Day 0) then after 2 weeks (Day 14) in order to assess allodynia. Only rats with a significant decrease in the minimal pressure (threshold) to trigger ipsilateral versus contralateral hindpaw withdrawal were included in the pharmacological groups; non-responders (noninjured animals) were considered an exclusion criteria. Each animal was used in only one experiment, after which it was sacrificed by CO2.

### REFERENCES

Zimmermann M. (1983). Ethical guidelines for investigations of experimental pain in conscious animals. Pain, 16, 109-10.
Dunham J., Hoedt-Schmidt S. and Kalbhen D. A. (1993). Prolonged effect of iodoacetate on articular cartilage and its modification by an anti-rheumatic drug. Int J Exp Pathol, 74, 283-9.

### Example A: Effects of Administration Compound 63.HCl (Example 1) and Tramadol on Osteoarthritic-Induced Allodynia

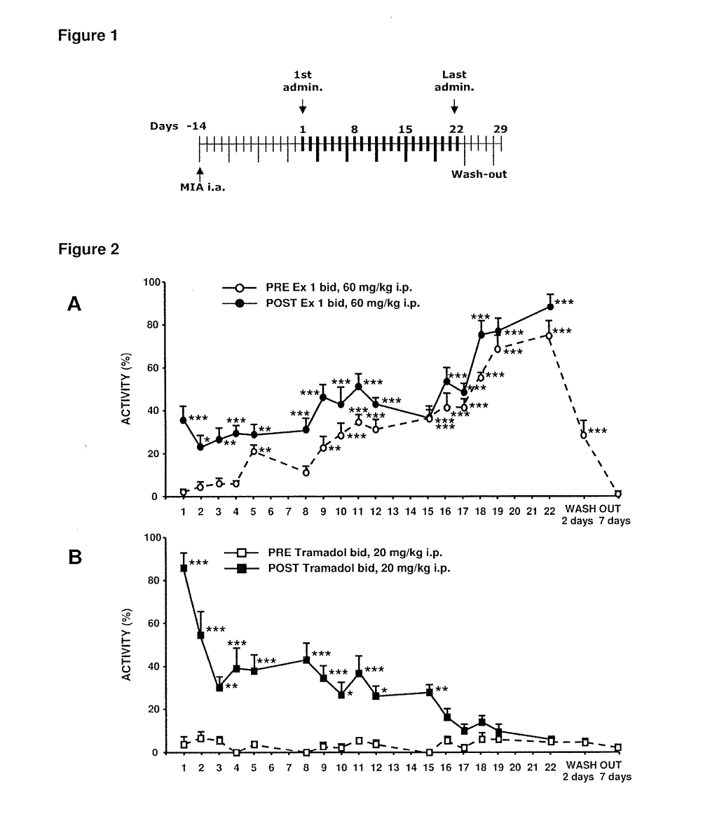

OA was induced by MIA injection at day −14 in the intra-patellar ligament of the right hindpaw. Sham animals received, in parallel, 50 μl of sterile saline solution. Two weeks later, allodynic rats were characterized by a marked decrease in the minimal pressure (threshold) to trigger withdrawal of the ipsilateral hindpaw (vs. the contralateral hindpaw) when stimulated by using von Frey monofilaments. From day 1 rats received chronic treatment (i.p, b.i.d.) for three weeks (until day 22) with Example 1 or Tramadol for three weeks, and treatment was then discontinued and mechanical allodynia evaluated after 2 days and 7 days (days 24 and 29, respectively) (wash-out period) (FIG. 1).

Two weeks after MIA injection into the knee, rats suffering from mechanical allodynia received a chronic treatment (3 weeks i.p, b.i.d.) with Example 1 (60 mg/kg, FIG. 2A) or Tramadol (20 mg/kg, FIG. 2B). Mechanical thresholds were determined before the first morning administration (open circles in FIG. 2A, open squares in FIG. 2B: PRE-Example 1 and PRE-Tramadol values, respectively) and then 30 min after treatments (full circles in FIG. 2A, full squares in FIG. 2B: POST-Example 1 and POST-Tramadol values, respectively). After three weeks (day 22), treatment was discontinued and mechanical allodynia was evaluated after 2 days and 7 days (wash-out period). Each point corresponds to the mean±S.E.M. of 10 (Example 1—and Tramadol-treated rats) vs. 9 (vehicle) independent determinations (one determination per rat). *p<0.05, **p<0.01, ***p<0.001, compared to corresponding values in vehicle-injected rats.

When comparing to Tramadol, example 1 (administered at 60 mg/kg intraperitoneally) is less efficacious than Tramadol (administered at 20 mg/kg intraperitoneally) following single treatment on day 1 (around 40% vs. 90% analgesia for example 1 and Tramadol, respectively), but efficacy of example 1 is higher than that of Tramadol following repeated daily treatments (on day 22, after three weeks of treatment, the analgesic effect was around 90% for example 1 vs. 10% for Tramadol). Interestingly, example 1 but not tramadol, progressively ameliorates the basal pain (PRE) found before daily treatments (80% reduction on day 22), which is consistent with a modification of mechanisms underlying pain.

Accordingly, the results allow concluding that Example 1, in contrast to Tramadol, does not induce analgesic tolerance (loss of analgesic efficacy following repeated treatment). On the contrary, it shows a surprising increase of activity with time following its repeated administration, which is associated with a progressive restoration back to normal of baseline nociceptive thresholds and strongly points to a disease-modifying effect. In fact, baseline pain before treatment (PRE; without treatment) ameliorates day by day and this effect parallels the increasing analgesic effect exerted by the compound (POST; 30 min after treatment), which indicates that the analgesic effect exerted by the compound (near 40% on day 1) is augmented by a disease-modifying effect that progressively ameliorates pain and allows an outstanding (90%) efficacy of the compound following repeated 22-days treatment. The disease-modifying effect exerted by the repeated treatment with example 1 is also evidenced after treatment discontinuation as pain on day 2 after discontinuation (wash out) is still reduced respect to the initial situation. On day 7 after treatment discontinuation (wash out) the pain went back to the initial situation, which indicates that the disease-modifying effect was indeed exerted by example 1 and that treatment with example 1 is required not only to modify the disease but also to maintain the modification.

### Example B: Effects of Administration of the Compound According to (Example 3) and Oxycodone on Osteoarthritic-Induced Allodynia

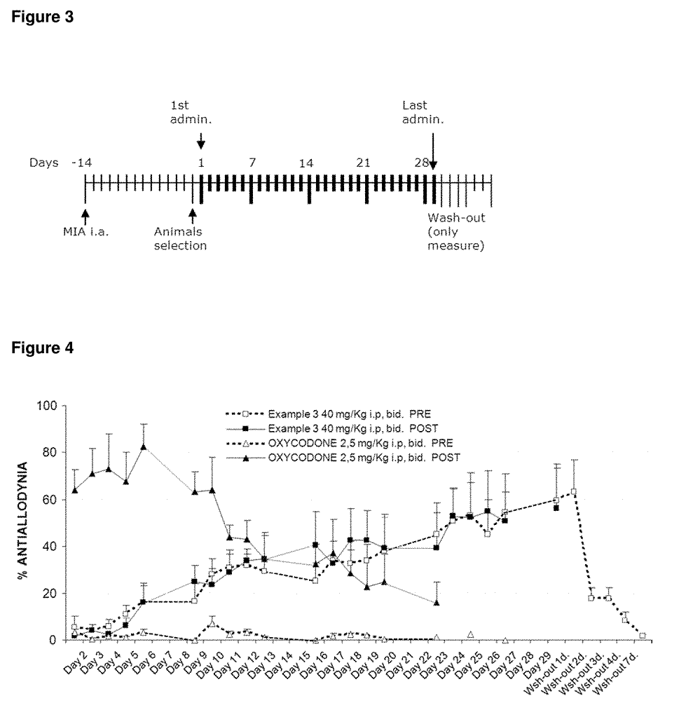

Oxycodone (open and full triangles in FIG. 4) was evaluated following the experimental protocol described previously (FIG. 1). Example 3 (Ex. 3) was evaluated according to the following protocol (FIG. 3):

Two weeks after MIA injection into the knee, rats suffering from mechanical allodynia received a chronic treatment with Ex.3 (i.p, b.i.d.) for four weeks (until day 29). After day 29, treatment was discontinued and mechanical allodynia was evaluated after 24 hours, 2 days and 7 days (wash-out period, days 30, 31 and 36, respectively) (wash-out period) (FIG. 1). Mechanical thresholds were determined before the first morning administration (open squares in FIG. 4) and then 30 min after treatments (full squares in FIG. 4). Each point corresponds to the mean±S.E.M. of 10 rats.

When comparing to oxycodone, Ex.3 (administered at 40 mg/kg intraperitoneally) is less efficacious than Oxycodone (administered at 2.5 mg/kg intraperitoneally) following single treatment on day 1 (around 5% vs. 65% analgesia for Ex.3 and oxycodone, respectively), but efficacy of Ex. 3 is higher than that of oxycodone following repeated daily treatments (on day 29, the analgesic effect was around 60% for Ex. 3 vs. 15% for Oxycodone, after four and three weeks of treatment, respectively). Interestingly, Ex.3 but not oxycodone, progressively ameliorates the basal pain (PRE) found before daily treatments (65% reduction on day 30), which is consistent with a modification of mechanisms underlying pain.

Therefore, it is concluded based on preclinical data in rats that compounds of the invention exert analgesic effects (and thus are useful for the treatment of osteoarthritis pain) and have disease modifying qualities in osteoarthritis (and thus are useful for the treatment of osteoarthritis and the prevention of osteoarthritis and pain due to osteoarthritis).

1-15. (canceled)

16. A method for the treatment or prevention of a condition selected from osteoarthritis and pain due to osteoarthritis in a subject in need thereof, comprising administration of a compound binding to the sigma-receptor.

17. The method according to claim 16, wherein the condition is pain due to osteoarthritis.

18. The method according to claim 17, wherein the pain is selected from acute and/or chronic pain due to osteoarthritis, including neuropathic pain, neuralgia, allodynia, causalgia, hyperalgesia, hyperesthesia, hyperpathia, neuritis or neuropathy secondary to surgical procedure.

19. The method according to claim 16, wherein the compound is selected from a sigma receptor antagonist, a neutral antagonist, an inverse agonist or a partial antagonist, and/or wherein the compound binds to the sigma-1 receptor subtype.

20. The method according to claim 16, wherein the compound is a compound of formula I: 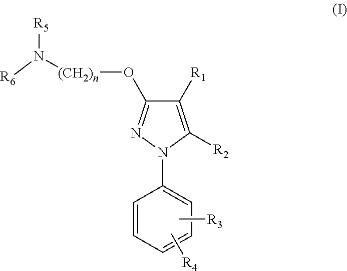
wherein
R₁ is selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or unsubstituted non-aromatic heterocyclyl, substituted or unsubstituted aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen;
R₂ is selected from the group consisting of hydrogen, substituted or unsubstituted alkyl, substituted or =substituted cycloalkyl, substituted or unsubstituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen;
R₃ and R₄ are independently selected from the group consisting of hydrogen, substituted or unsubstituted alkyl, substituted or unsubstituted cycloalkyl, substituted or =substituted alkenyl, substituted or unsubstituted aryl, substituted or unsubstituted arylalkyl, substituted or unsubstituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen, or together with the phenyl ring to which they are attached, form an optionally substituted fused ring system;
R₅ and R₆ are independently selected from the group consisting of hydrogen, substituted or unsubstituted alkyl, substituted or unsubstituted cycloalkyl, substituted or unsubstituted alkenyl, substituted or unsubstituted aryl, substituted or unsubstituted arylalkyl, substituted or =substituted, aromatic or non-aromatic heterocyclyl, substituted or unsubstituted heterocyclylalkyl, —COR₈, —C(O)OR₈, —C(O)NR₈R₉, —CH═NR₈, —CN, —OR₈, —OC(O)R₈, —S(O)t-R₈, —NR₈R₉, —NR₈C(O)R₉, —NO₂, —N═CR₈R₉, and halogen, or, together with the nitrogen atom to which they are attached, form a substituted or unsubstituted, aromatic or non-aromatic heterocyclyl group;
n is 1, 2, 3, 4, 5, 6, 7, or 8;
t is 1, 2, or 3;
R₈ and R₉ are each independently selected from hydrogen, substituted or unsubstituted alkyl, substituted or unsubstituted cycloalkyl, substituted or unsubstituted alkenyl, substituted or unsubstituted aryl, substituted or unsubstituted, aromatic or non-aromatic heterocyclyl, substituted or unsubstituted alkoxy, substituted or unsubstituted aryloxy, and halogen;
or a pharmaceutically acceptable salt, isomer or solvate thereof.

21. The method according to claim 20, wherein R₁ is hydrogen.

22. The method according to claim 20, wherein R₂ is H or alkyl; and/or
R₃ and R₄ together with the phenyl form a naphthyl group.

23. The method according to claim 22, wherein R₂ is H or methyl.

24. The method according to claim 20, wherein n is 2, 3, or 4; and/or
R₅ and R₆, together with the nitrogen atom to which they are attached, form a morpholine-4-yl group.

25. The method according to claim 24, wherein n is 2.

26. The method according to claim 16, wherein the compound is selected from 4-{2-[5-Methyl-1-(naphthalen-2-yl)-1H-pyrazol-3-yloxy]ethyl}morpholine, or a pharmaceutically acceptable salt or solvate thereof, and 4-{2-[5-Methyl-1-(naphthalen-2-yl)-1H-pyrazol-3-yloxy]ethyl}morpholine hydrochloride or a solvate thereof.

27. The method according to claim 16, wherein the compound is a compound of formula II: 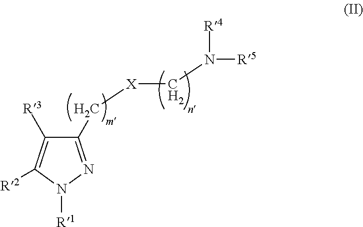
wherein
R′¹ represents substituted or unsubstituted aromatic or non-aromatic heterocyclyl, substituted or unsubstituted aryl, or substituted or unsubstituted cycloalkyl;
R′² and R′³, which may be identical or different, represent a hydrogen atom, F, Cl, Br, I, CF₃, OH, SH, NH₂, CN, substituted or unsubstituted alkyl, substituted or unsubstituted alkenyl, substituted or unsubstituted alkoxy, substituted or unsubstituted cycloalkyl, substituted or unsubstituted aryl, substituted or unsubstituted, aromatic or non-aromatic heterocyclyl, substituted or unsubstituted cycloalkylalkyl, substituted or unsubstituted arylalkyl, substituted or unsubstituted, aromatic or non-aromatic heterocyclylalkyl, a (C═O)—R′⁷ group, a (C═O)—O—R′⁸ group, a S(O)ₜ—R′⁹ group, or a (C═O)—NR′¹⁰R′¹¹ group;
R′⁴ and R′⁵, which may be identical or different, represent a hydrogen atom, substituted or unsubstituted alkyl, substituted or unsubstituted alkenyl, substituted or unsubstituted alkoxy, substituted or unsubstituted cycloalkyl, substituted or unsubstituted aryl, substituted or unsubstituted, aromatic or non-aromatic heterocyclyl, substituted or unsubstituted cycloalkylalkyl, substituted or unsubstituted arylalkyl, substituted or unsubstituted, aromatic or non-aromatic heterocyclylalkyl, a (C═O)—R′⁷ group, a (C═O)—O—R′⁸ group, an S(O)_{t′}—R′⁹ group, or a (C═O)—NR′¹⁰R′¹¹ group;
or
together with the nitrogen atom to which they are attached, form a substituted or unsubstituted, aromatic or non-aromatic heterocyclyl group;
X represents an oxygen atom or a CH—R′¹² group, wherein R′¹² is selected from H, CH₃, SH, OH, NH₂, CF₃, Cl, F, Br, I, and CN;
m′ is selected from 1, 2, 3 and 4;
n′ is selected from 1, 2, 3 and 4;
t′ is selected from 1, 2 and 3;
R′⁷, R′⁸, R′⁹, R′¹⁰ and R′¹¹, which may be identical or different, represent a hydrogen atom, substituted or unsubstituted C₁₋₆ alkyl, substituted or unsubstituted C₁₋₆ alkenyl, substituted or unsubstituted C₁₋₆ alkoxy, substituted or unsubstituted cycloalkyl, substituted or unsubstituted aryl, substituted or unsubstituted, aromatic or non-aromatic heterocyclyl, substituted or unsubstituted cycloalkylalkyl, substituted or unsubstituted arylalkyl, substituted or unsubstituted, aromatic or non-aromatic heterocyclylalkyl;
or a pharmaceutically acceptable salt, isomer or solvate thereof.

28. The method according to claim 27, wherein R′¹ is selected from a 5- to 10 membered substituted or unsubstituted, aromatic or non-aromatic heterocyclyl group which may contain N, O or S as a ring member; a 5- to 10 membered substituted or unsubstituted aryl group, and a 5- to 10 membered substituted or unsubstituted cycloalkyl group.

29. The method according to claim 28, wherein R′¹ is selected from substituted or unsubstituted cyclopentyl, substituted or =substituted cyclohexyl, substituted or unsubstituted phenyl, substituted or unsubstituted naphthyl, substituted or unsubstituted thiophene, substituted or unsubstituted benzothiophene, substituted or unsubstituted benzofuran, substituted or unsubstituted pyridine, and substituted or unsubstituted quinoline.

30. The method according to claim 27, wherein R′² and R′³ are independently selected from H and substituted or unsubstituted C₁₋₆ alkyl.

31. The method according to claim 31, wherein R′² and R′³ are independently selected from H and methyl.

32. The method according to claim 27, wherein R′⁴ and R′⁵, together with the nitrogen atom to which they are attached, form a substituted or unsubstituted heterocyclyl group.

33. The method according to claim 32, wherein R′⁴ and R′³, together with the nitrogen atom to which they are attached, form a morpholine-4-yl group, a piperidine-4-yl group, pyrrolidine-4-yl group or a piperazine-4-yl group.

34. The method according to claim 27, wherein m′ and n′ are independently selected from 1 and 2; and/or
X represents an oxygen atom or a —CH₂— group.

35. The method according to claim 27, wherein the compound is selected from 1-(4-(2-((1-(3,4-difluorophenyl)-1H-pyrazol-3-yl)methoxy)ethyl)piperazin-1-yl)ethanone, or a pharmaceutically acceptable salt or solvate thereof, and 1-(4-(2-((1-(3,4-difluorophenyl)-1H-pyrazol-3-yl)methoxy)ethyl)piperazin-1-yl)ethanone hydrochloride or a solvate thereof.

36. The method according to claim 16, wherein the compound is selected from 1-(4-(2-((1-(3-difluorophenyl)-1H-pyrazol-3-yl)methoxy)ethyl)piperazin-1-yl)ethanone, or a pharmaceutically acceptable salt or solvate thereof; 1-(4-(2-((1-(3,4-difluorophenyl)-1H-pyrazol-3-yl)methoxy)ethyl)piperazin-1-yl)ethanone hydrochloride or a solvate thereof;
4-{2-[5-Methyl-1-(naphthalen-2-yl)-1H-pyrazol-3-yloxy]ethyl}morpholine, or a pharmaceutically acceptable salt or solvate thereof; and 4-{2-[5-Methyl-1-(naphthalen-2-yl)-1H-pyrazol-3-yloxy]ethyl}morpholine hydrochloride or a solvate thereof.
